(19) United States
(12) Patent Application Publication
(10) Pub. No.: US 2024/0363240 A1
(43) Pub. Date: Oct. 31, 2024

(54) MANAGEMENT OF MEDICATION DISPENSER USING TELECOMMUNICATIONS SYSTEM

(71) Applicant: T-Mobile USA, Inc., Bellevue, WA (US)

(72) Inventors: Hajer Alarak, Lynnwood, WA (US); Blaine Virgil Anderson, Tacoma, WA (US); Blake Louis Bollinger, Baton Rouge, LA (US); Shelly Dhiman, Snoqualmie, WA (US); Jaemin Kim, Olympia, WA (US); Meera Mohandas, Redmond, WA (US); Nhan Thy Nguyen, Bellevue, WA (US); Zhifu Pei, Bellevue, WA (US)

(21) Appl. No.: 18/308,434

(22) Filed: Apr. 27, 2023

(51) Int. Cl. G16H 40/67; G16H 20/13

(52) U.S. Cl. CPC G16H 40/67; G16H 20/13

(57) ABSTRACT

A server system receives sensor data generated by one or more sensors of a medication dispenser. The medication dispenser can include multiple openable compartments for holding medicines. The sensor data includes sensor data generated by a sensor of the multiple sensors. The sensor detects the opening of a lid of a respective compartment of the multiple compartments. The server system determines whether the lid of the respective compartment has been opened during a particular time period, based on the sensor data. The respective compartment holds medicines to be consumed by a user of the medication dispenser during the particular time period. In response to a determination that the lid of the respective compartment has not been opened during the particular time period, the server system causes a wireless device to output a notification indicating that the lid of the respective compartment has not been opened during the particular time period.

### BACKGROUND

Electronic medication dispensers (e.g., electronic pill boxes or electronic pill dispensers) are standalone devices for assisting patients to adhere to their medication regimes (e.g., prescription medication regimes). Such dispensers are important for the safe consumption of prescription medications. The dispensers can include multiple departments for storing medications to be taken at different times of the day on different days of the week. The dispensers can allow a patient to pre-fill the different compartments for a certain time period (e.g., a week or a day) allowing the patient to take the correct amounts of medication at appropriate times. Electronic medication dispensers can also track the consumption of medication and, for example, provide alerts to the patient.

### BRIEF DESCRIPTION OF THE DRAWINGS

Detailed descriptions of implementations of the present technology will be described and explained through the use of the accompanying drawings.

FIG. 1 is a block diagram that illustrates a wireless communications system that can implement aspects of the present technology.

FIG. 2 is a block diagram that illustrates a system for the management of medication dispensing.

FIG. 3 is a block diagram that illustrates an electronic container.

FIGS. 4A-4C are schematic diagrams that illustrate a graphical user interface (GUI) for the management of an electronic medication dispenser.

FIG. 5 is a flow diagram that illustrates processes for the management of medication dispensing.

FIG. 6 is a block diagram that illustrates an example of a computer system in which at least some operations described herein can be implemented.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

The disclosed technology relates to a system, associated with a telecommunications network, for managing medication dispensing. The system can include a server system configured to communicate with an electronic medication dispenser (e.g., an electronic medication container or an electronic pill box) and one or more wireless devices. The electronic medication dispenser can include sensors that track a patient's interactions with the medication dispenser. For example, the electronic medication dispenser can include a sensor for detecting the opening of a compartment of the medication dispenser and removal of medication stored in the compartment. The server system can receive time-stamped sensor data generated by the sensor. Based on the sensor data, the server system can determine whether the compartment of the medication dispenser was opened during a particular time period of a day and whether the medication stored in the compartment was removed. The server system can communicate instructions to the one or more wireless devices based on the sensor data. For example, if the server system determined that the patient has not removed the medication during a particular time period, the server system can cause a wireless device to display a notification (e.g., a reminder message or an alert).

In one implementation, the electronic medication dispenser is configured to connect to the server system over the telecommunications network and/or to connect directly to the wireless device over a local network. The electronic medication dispenser and the wireless device are associated with a common subscriber having one or more subscriptions to a common carrier of the telecommunications network. The electronic medication dispenser and the wireless device are configured to connect directly to each other over the local network and configured to connect to the server system on different channels over the telecommunications network. Moreover, communications from the electronic medication dispenser to the server system are routed through or by the wireless device.

For example, the electronic medication dispenser can be paired to the wireless device and connect directly over Bluetooth or Wi-Fi when in proximity of each other. As such, communications between the electronic medication dispenser and the server system are relayed through the wireless device. The electronic medication dispenser can also communicate with the server system over the telecommunications network by routing communications through the wireless device. As such, the wireless device remains synced with the electronic medication dispenser but without needing to be in proximity to each other such that a user of the electronic medication dispenser does not need to carry both devices to receive timely notifications for managing medication dispensing.

In one example, the server system can receive sensor data generated and/or collected by one or more sensors of the electronic medication dispenser from the electronic medication dispenser. The electronic medication dispenser can include multiple openable compartments for holding medicines. The sensor data can include first sensor data generated by a first sensor of the multiple sensors. The first sensor can detect the opening or closing of a lid of a respective compartment of the multiple compartments of the electronic medication dispenser. The server system can determine whether the lid of the respective compartment has been opened during a particular time period based on the first sensor data. The respective compartment can be configured to hold medicines to be consumed by a user of the electronic medication dispenser during the particular time period. In response to a determination that the lid of the respective compartment has not been opened during the particular time period, the server system can cause the wireless device to output a notification indicating that the lid of the respective compartment has not been opened during the particular time period.

In another example, a server system is configured to be in communication with a wireless device and a smart container. The server system can receive sensor data generated by one or more sensors of the smart container from the smart container. The smart container can include an openable lid. The sensor data can include first sensor data generated by a first sensor of the one or more sensors. The first sensor can detect opening of the lid of the smart container. The server system can determine whether the lid of the smart container has been opened based on the first sensor data. In accordance with a determination that the lid of the smart container has been opened, the server system can cause the wireless device to perform an action.

In another example, a method for managing medication dispensing by a server system includes receiving sensor data from an electronic medication dispenser. The sensor data can be generated by one or more sensors of the electronic medication dispenser. The electronic medication dispenser can include multiple openable compartments for holding medicines. The sensor data can include first sensor data generated by a first sensor of the multiple sensors. The first sensor can detect the opening of a lid of a respective compartment of the multiple compartments of the electronic medication dispenser. The server system can determine whether the lid of the respective compartment has been opened during a particular time period based on the first sensor data. The respective compartment can be configured to hold medicines to be consumed by a user of the electronic medication dispenser during the particular time period. In response to a determination that the lid of the respective compartment has not been opened during the particular time period, the server system can cause a wireless device to output a notification indicating that the lid of the respective compartment has not been opened during the particular time period.

The description and associated drawings are illustrative examples and are not to be construed as limiting. This disclosure provides certain details for a thorough understanding and enables description of these examples. One skilled in the relevant technology will understand, however, that the invention can be practiced without many of these details. Likewise, one skilled in the relevant technology will understand that the invention can include well-known structures or features that are not shown or described in detail to avoid unnecessarily obscuring the descriptions of examples.

### Wireless Communications System

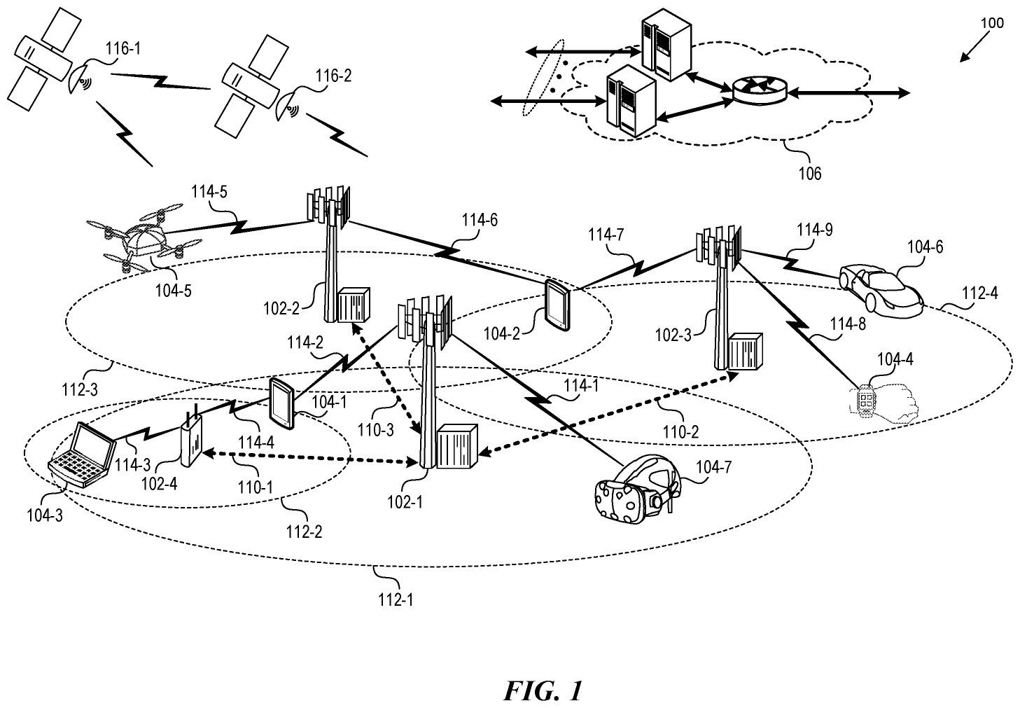

FIG. 1 is a block diagram that illustrates a wireless telecommunications network 100 (“network 100”) in which aspects of the disclosed technology are incorporated. The network 100 includes base stations 102-1 through 102-4 (also referred to individually as “base station 102” or collectively as “base stations 102”). A base station is a type of network access node (NAN) that can also be referred to as a cell site, a base transceiver station, or a radio base station. The network 100 can include any combination of NANs including an access point, radio transceiver, gNodeB (gNB), NodeB, eNodeB (eNB), Home NodeB or Home eNodeB, or the like. In addition to being a wireless wide area network (WWAN) base station, a NAN can be a wireless local area network (WLAN) access point, such as an Institute of Electrical and Electronics Engineers (IEEE) 802.11 access point.

The NANs of a network 100 formed by the network 100 also include wireless devices 104-1 through 104-7 (referred to individually as “wireless device 104” or collectively as “wireless devices 104”) and a core network 106. The wireless devices 104-1 through 104-7 can correspond to or include network 100 entities capable of communication using various connectivity standards. For example, a 5G communication channel can use millimeter wave (mmW) access frequencies of 28 GHz or more. In some implementations, the wireless device 104 can operatively couple to a base station 102 over a long-term evolution/long-term evolution-advanced (LTE/LTE-A) communication channel, which is referred to as a 4G communication channel.

The core network 106 provides, manages, and controls security services, user authentication, access authorization, tracking, Internet Protocol (IP) connectivity, and other access, routing, or mobility functions. The base stations 102 interface with the core network 106 through a first set of backhaul links (e.g., S1 interfaces) and can perform radio configuration and scheduling for communication with the wireless devices 104 or can operate under the control of a base station controller (not shown). In some examples, the base stations 102 can communicate with each other, either directly or indirectly (e.g., through the core network 106), over a second set of backhaul links 110-1 through 110-3 (e.g., X1 interfaces), which can be wired or wireless communication links.

The base stations 102 can wirelessly communicate with the wireless devices 104 via one or more base station antennas. The cell sites can provide communication coverage for geographic coverage areas 112-1 through 112-4 (also referred to individually as “coverage area 112” or collectively as “coverage areas 112”). The geographic coverage area 112 for a base station 102 can be divided into sectors making up only a portion of the coverage area (not shown). The network 100 can include base stations of different types (e.g., macro and/or small cell base stations). In some implementations, there can be overlapping geographic coverage areas 112 for different service environments (e.g., Internet-of-Things (IoT), mobile broadband (MBB), vehicle-to-everything (V2X), machine-to-machine (M2M), machine-to-everything (M2X), ultra-reliable low-latency communication (URLLC), machine-type communication (MTC), etc.).

The network 100 can include a 5G network 100 and/or an LTE/LTE-A or other network. In an LTE/LTE-A network, the term eNB is used to describe the base stations 102, and in 5G new radio (NR) networks, the term gNBs is used to describe the base stations 102 that can include mmW communications. The network 100 can thus form a heterogeneous network 100 in which different types of base stations provide coverage for various geographic regions. For example, each base station 102 can provide communication coverage for a macro cell, a small cell, and/or other types of cells. As used herein, the term “cell” can relate to a base station, a carrier or component carrier associated with the base station, or a coverage area (e.g., sector) of a carrier or base station, depending on context.

A macro cell generally covers a relatively large geographic area (e.g., several kilometers in radius) and can allow access by wireless devices that have service subscriptions with a wireless network 100 service provider. As indicated earlier, a small cell is a lower-powered base station, as compared to a macro cell, and can operate in the same or different (e.g., licensed, unlicensed) frequency bands as macro cells. Examples of small cells include pico cells, femto cells, and micro cells. In general, a pico cell can cover a relatively smaller geographic area and can allow unrestricted access by wireless devices that have service subscriptions with the network 100 provider. A femto cell covers a relatively smaller geographic area (e.g., a home) and can provide restricted access by wireless devices having an association with the femto unit (e.g., wireless devices in a closed subscriber group (CSG), wireless devices for users in the home). A base station can support one or multiple (e.g., two, three, four, and the like) cells (e.g., component carriers). All fixed transceivers noted herein that can provide access to the network 100 are NANs, including small cells.

The communication networks that accommodate various disclosed examples can be packet-based networks that operate according to a layered protocol stack. In the user plane, communications at the bearer or Packet Data Convergence Protocol (PDCP) layer can be IP-based. A Radio Link Control (RLC) layer then performs packet segmentation and reassembly to communicate over logical channels. A Medium Access Control (MAC) layer can perform priority handling and multiplexing of logical channels into transport channels. The MAC layer can also use Hybrid ARQ (HARQ) to provide retransmission at the MAC layer, to improve link efficiency. In the control plane, the Radio Resource Control (RRC) protocol layer provides establishment, configuration, and maintenance of an RRC connection between a wireless device 104 and the base stations 102 or core network 106 supporting radio bearers for the user plane data. At the physical (PHY) layer, the transport channels are mapped to physical channels.

Wireless devices can be integrated with or embedded in other devices. As illustrated, the wireless devices 104 are distributed throughout the wireless telecommunications network 100, where each wireless device 104 can be stationary or mobile. For example, wireless devices can include handheld mobile devices 104-1 and 104-2 (e.g., smartphones, portable hotspots, tablets, etc.); laptops 104-3; wearables 104-4; drones 104-5; vehicles with wireless connectivity 104-6; head-mounted displays with wireless augmented reality/virtual reality (AR/VR) connectivity 104-7; portable gaming consoles; wireless routers, gateways, modems, and other fixed-wireless access devices; wirelessly connected sensors that provides data to a remote server over a network; IoT devices such as wirelessly connected smart home appliances, etc.

A wireless device (e.g., wireless devices 104-1, 104-2, 104-3, 104-4, 104-5, 104-6, and 104-7) can be referred to as a user equipment (UE), a customer premise equipment (CPE), a mobile station, a subscriber station, a mobile unit, a subscriber unit, a wireless unit, a remote unit, a handheld mobile device, a remote device, a mobile subscriber station, terminal equipment, an access terminal, a mobile terminal, a wireless terminal, a remote terminal, a handset, a mobile client, a client, or the like.

A wireless device can communicate with various types of base stations and network 100 equipment at the edge of a network 100 including macro eNBs/gNBs, small cell eNBs/gNBs, relay base stations, and the like. A wireless device can also communicate with other wireless devices either within or outside the same coverage area of a base station via device-to-device (D2D) communications.

The communication links 114-1 through 114-9 (also referred to individually as “communication link 114” or collectively as “communication links 114”) shown in network 100 include uplink (UL) transmissions from a wireless device 104 to a base station 102, and/or downlink (DL) transmissions from a base station 102 to a wireless device 104. The downlink transmissions can also be called forward link transmissions while the uplink transmissions can also be called reverse link transmissions. Each communication link 114 includes one or more carriers, where each carrier can be a signal composed of multiple sub-carriers (e.g., waveform signals of different frequencies) modulated according to the various radio technologies. Each modulated signal can be sent on a different sub-carrier and carry control information (e.g., reference signals, control channels), overhead information, user data, etc. The communication links 114 can transmit bidirectional communications using frequency division duplex (FDD) (e.g., using paired spectrum resources) or time division duplex (TDD) operation (e.g., using unpaired spectrum resources). In some implementations, the communication links 114 include LTE and/or mmW communication links.

In some implementations of the network 100, the base stations 102 and/or the wireless devices 104 include multiple antennas for employing antenna diversity schemes to improve communication quality and reliability between base stations 102 and wireless devices 104. Additionally or alternatively, the base stations 102 and/or the wireless devices 104 can employ multiple-input, multiple-output (MIMO) techniques that can take advantage of multi-path environments to transmit multiple spatial layers carrying the same or different coded data.

In some examples, the network 100 implements 6G technologies including increased densification or diversification of network nodes. The network 100 can enable terrestrial and non-terrestrial transmissions. In this context, a Non-Terrestrial Network (NTN) is enabled by one or more satellites such as satellites 116-1 and 116-2 to deliver services anywhere and anytime and provide coverage in areas that are unreachable by any conventional Terrestrial Network (TN). A 6G implementation of the network 100 can support terahertz (THz) communications. This can support wireless applications that demand ultrahigh quality of service requirements and multi-terabits per second data transmission in the 6G and beyond era, such as terabit-per-second backhaul systems, ultrahigh-definition content streaming among mobile devices, AR/VR, and wireless high-bandwidth secure communications. In another example of 6G, the network 100 can implement a converged Radio Access Network (RAN) and Core architecture to achieve Control and User Plane Separation (CUPS) and achieve extremely low user plane latency. In yet another example of 6G, the network 100 can implement a converged Wi-Fi and Core architecture to increase and improve indoor coverage.

### Management of Medication Dispensing

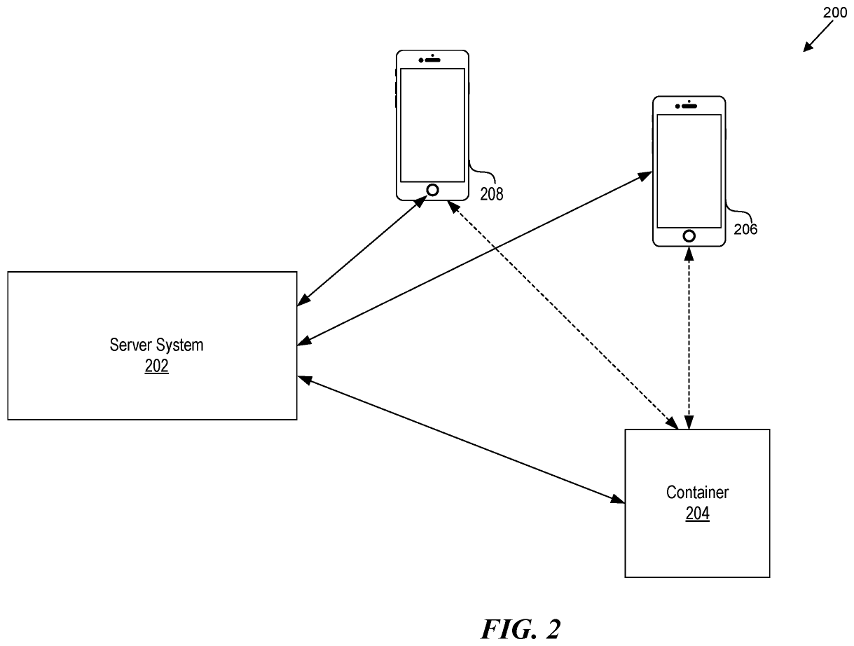

FIG. 2 is a block diagram that illustrates a system 200 for the management of medication dispensing. The system 200 includes a server system (e.g., a server system 202), an electronic container (e.g., a container 204), and one or more wireless devices (e.g., wireless devices 206 and 208).

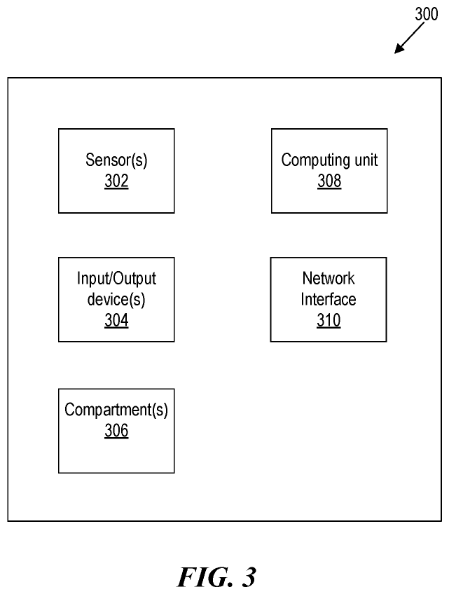

The container 204 can be a medication dispenser (e.g., an electronic pill box or a smart medication dispenser) that is configured to assist patients with safe and accurate medication intake. In particular, the container 204 can be useful when a patient is on a prescription regime including multiple medications that are to be taken multiple times a day. The container 204 is configured to communicate wirelessly with the server system 202 (e.g., via the Internet). For example, the container 204 can include one or more sensors for collecting sensor data associated with the container 204. The container 204 can be configured to communicate the sensor data to the server system 202. In some implementations, the container 204 can also be in communication with the wireless devices 206 and 208 (e.g., via a Wi-Fi connection, or a Bluetooth connection). The container 204 is described in detail with respect to FIG. 3.

The server system 202 can be associated with a wireless service provider (e.g., a provider for the wireless telecommunications network 100 in FIG. 1). The server system 202 can be configured to be in wireless communication with the container 204 and the wireless devices 206 and 208. For example, the server system 202 can receive the sensor data from the container and process the data. The server system 202 can also send instructions or indications to the container 204. The server system 202 can also be in wireless communication with the wireless device 206 and/or the wireless device 208. For example, the server system 202 can communicate instructions and/or indications to a software application operating on the wireless device 206 and/or the wireless device 208 and cause the software application to perform actions. The server system 202 can also receive instructions and/or indications from the wireless devices 206 and/or 208.

The wireless devices 206 and 208 can be any types of wireless devices configured to communicate with the server system 202. For example, the wireless devices 206 and 208 can be individually selected from a smartphone, a tablet computer, a laptop computer, or a personal computer. The wireless devices 206 and 208 include one or more software applications associated with the system 200. The one or more software applications can enable providing messages and information to a user of a respective wireless device. The one or more software applications can also enable receiving user inputs from the user of the respective wireless device and forwarding instructions and/or indications to the server system in response to the user inputs. The wireless devices 206 and 208 can be associated with patients, caregivers, health care providers, pharmacies, etc. For example, the wireless device 206 can be associated with a patient who is using the container 204 or a caregiver of such patient, and the wireless device 208 is associated with the patient's health care provider (e.g., a hospital, a nurse, or a physician) or the patient's pharmacy.

In some implementations, the container 204 is configured to connect to the server system 202 over the telecommunications network (e.g., the wireless telecommunications network 100 in FIG. 1) and/or to connect directly to the wireless device over a local network (e.g., a Bluetooth or Wi-Fi network). The container 204 and the wireless devices 206 and 208 can be associated with a common subscriber having one or more subscriptions to a common carrier of the telecommunications network. The container 204 and the wireless devices 206 and 208 can be configured to connect directly to each other over the local network and configured to connect to the server system 202 on different channels over the telecommunications network. Moreover, communications from the container 204 to the server system 202 are routed through or by the wireless device.

As an example, the container 204 can be paired to the wireless device 206 and connect directly over the Bluetooth or Wi-Fi local network when in proximity of each other (e.g., within a range of the local network). As such, communications between the container 204 and the server system 202 are relayed through the wireless device 206. The container 204 can also communicate with the server system 202 over the telecommunications network by routing communications through the wireless device 206. As such, the wireless device can remain synced with the container 204 but without needing to be in proximity to each other. For example, the system 200 can thereby allow a patient (or a caregiver) to use their wireless device to communicate with their medical dispenser without needing to carry both devices to receive timely notifications for managing medication dispensing.

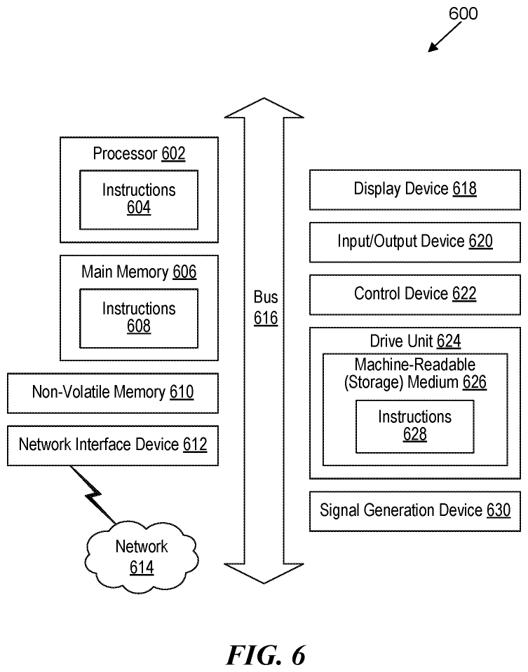

FIG. 3 is a block diagram that illustrates an electronic container 300. In some implementations, the container 300 corresponds to the container 204 of the system 200 in FIG. 2. In some implementations, the container 300 can be a medication dispenser. The container 300 can include one or more sensors (e.g., sensors 302), one or more input and one or more output devices (e.g., input and output devices 304), one or more compartments (e.g., compartments 306), a computing unit (e.g., a computing unit 308), and a network interface (e.g., a network interface 310). In some implementations, the container 300 corresponds to or includes portions of a computer device (e.g., a computing system 600 described with respect to FIG. 6).

As described with respect to FIG. 2, in some implementations the container 300 is a medication dispenser. The one or more compartments 306 can be configured for holding medications. The medications can be in the form of tablets, pills, capsules, or soft gels. The container 300 can include a sufficient number of compartments to hold medications to be consumed multiple times a day for seven to 14 days. As an example, the container can include 21 compartments for storing medications to be consumed three times a day (morning, midday, and evening) over seven days. The compartments 306 are openable either individually or jointly. For example, each of the compartments 306 can include a lid (e.g., a cover or a top) that can be opened while other compartments stay covered. The lids of the compartments can be opened electronically or manually.

The sensors 302 can be configured to collect sensor data associated with the container 300. The sensors 302 can include one or more of a temperature sensor, a global positioning system (GPS) tracker, an image sensor, a weight sensor, and a motion sensor. The temperature sensor can be configured to measure the temperature (e.g., ambient temperature) of the container 300. The GPS tracker can be configured to detect location of the container 300. The image sensor can be configured to collect image data that depicts, for example, whether a lid of a respective compartment 306 is closed or open or whether the respective compartment includes medicines. The weight sensor can be configured to measure the weight of medication stored inside a respective compartment 306. For example, the measured weight can provide an indication of whether the respective compartment 306 contains medications or not (i.e., whether a patient removed the medications from the respective compartment). The optical sensor (e.g., an ultraviolet (UV) light sensor or an infrared (IR) light sensor) can be configured to detect whether a lid of a respective compartment 306 is open or closed. The motion sensor can be configured to detect movement of the container 300 and/or the movement of a lid of a respective compartment 306.

The input and/or output devices 304 can include any devices and user interfaces for allowing a user to interact with the container 300. The output devices can include, for example, visual (e.g., a light indicator or a display) and audio (e.g., a speaker) output devices. The input devices can include buttons, knobs, touch screens, etc., that allow a user to interact with the container 300.

In some implementations, the container 300 is a smart device (e.g., referred to as a smart container). A smart device refers to a device configured for communicating with other devices or server systems via one or more wireless protocols while being capable of operating at least to some extent interactively and autonomously. The container 300 can thereby include a network interface 310 and a computing unit 308. In some implementations, the network interface 310 can correspond to a network interface device 612 described with respect to FIG. 6. For example, the network interface 310 can allow the container 300 to connect to a server system (e.g., the server system in FIG. 2) over a telecommunications network (e.g., the wireless telecommunications network 100 in FIG. 1) and/or to connect directly to one or more wireless device over a local network (e.g., connecting to the wireless devices 206 and/or 208 over a Bluetooth or Wi-Fi network). The network interface 310 can enable the computing unit 308 to communicate with the server system 202 and optionally the wireless devices 206 and/or 208. The computing unit 308 can include, for example, a processor, memory, and/or controller. In some implementations, the processor can correspond to a processor 602, the memory can correspond to memory 606 and/or memory 610, and the controller can correspond to a control device 622 of the computing system 600 described with respect to FIG. 6. The computing unit 308 can enable operations of the container 300 such as operation of the sensors 302, the input and/or output devices 304, the compartments 306, and the network interface 310.

In some implementations, the container 300 can be configured to store items other than medications. For example, the container 300 can be configured to store sensitive or valuable items. Such sensitive or valuable items can include, for example, money, jewelry, documents, personal artifacts, artwork, or electronics. The container 300 can enable tracking of the contents stored inside the container 300. For example, the container 300 can be used to detect the location of the items when inside the container 300, whether a lid of a compartment 306 of the container 300 has been opened, and whether any items have been removed from the container 300. As another example, a user can be alerted if the container 300 has been opened (e.g., items stored inside the container 300 have been tampered with or stolen).

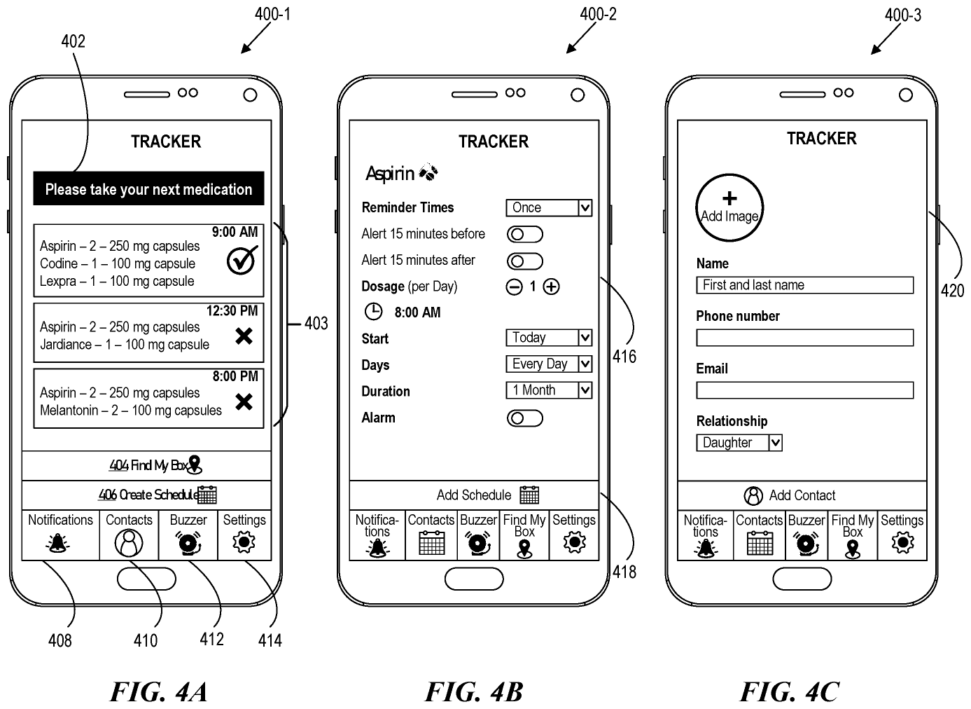

FIGS. 4A-4C are schematic diagrams that illustrate a GUI 400 for the management of an electronic medication dispenser. FIG. 4A illustrates a GUI 400-1 (e.g., a home page GUI), FIG. 4B illustrates a GUI 400-2 (e.g., a GUI for generating a schedule), and FIG. 4C illustrates a GUI 400-3 (e.g., a GUI for adding and storing user contact information). The GUIs 400-1, 400-2, and 400-3 are collectively referred to as the GUI 400. The GUI 400 can be displayed on a display of a wireless device (e.g., the wireless devices 206 and 208 in FIG. 2). The GUI 400 can be associated with a software application operating on the wireless device that is configured to communicate with the server system (e.g., the server system 202 in FIG. 2).

In FIG. 4A, the GUI 400-1 can include a portion 402 for displaying messages and a portion 403 for displaying information related to one or more scheduled times for taking medications. The messages displayed in the portion 402 can include reminders and alerts. The messages can be pop-up or push-on notifications that are displayed occasionally. For example, in FIG. 4A the portion 402 includes a message “Please take your next medication” that operates as a reminder for a patient to take his or her medicines. The portion 403 includes three subregions indicating a planned medication regime at three different times of the day (e.g., at 9:00 AM, at 12:30 PM, and at 8:00 PM). The subregions can identify the type and amount of medicine that should be taken at the respective times of the day. The regions can further include a symbol indicating whether the medication has been taken or not (e.g., a check symbol indicating that the medication was taken at 9:00 AM and a cross symbol indicating that the medication has not been taken at 12:30 PM). For example, the server system 202 in FIG. 2 can determine that the medication was taken at 9:00 AM based on sensor data received from the container 204. The server system can send the wireless device an indication that the medication was taken. In response to receiving the indication, the wireless device displays a check symbol in the portion 403 of the GUI 400-1.

The GUI 400-1 can also include multiple affordances. An affordance refers to an object displayed on a GUI that is associated with a particular operation and causes a computer system to perform the particular operation in response to a user interaction on the affordance (e.g., by a user input such as a tap, a swipe, or a press). The object can include a text, symbol, image, or space for entering user input (e.g., an affordance for entering a search term). Affordances enable convenient and intuitive ways for a user to operate a system via a GUI. The GUI 400-1 can include, for example, an affordance 404 for locating the container associated with the GUI 400, an affordance 406 for generating a schedule that indicates medication intake times, an affordance 408 for displaying notifications, an affordance 410 for displaying contact information, an affordance 412 for providing alerts, and an affordance 414 for displaying settings. For example, a user can grant their caretaker the ability to press the affordance 412 that triggers an output device (e.g., a buzzer) of the container 204 to alert the user. The caretaker can use this function to remind the user in an instance that the caretaker notices that the user has not taken his or her medication. As another example, the buzzer can be preset to automatically sound in an instance that the container 204 has not been opened at a scheduled medication time (after a configurable grace period). If a user is supposed to take a medication at 9:00 am but for some reason misses the scheduled time, the buzzer would sound automatically at 9:30 am to alert the user to take their medication (assuming the grace period is set to 30 minutes).

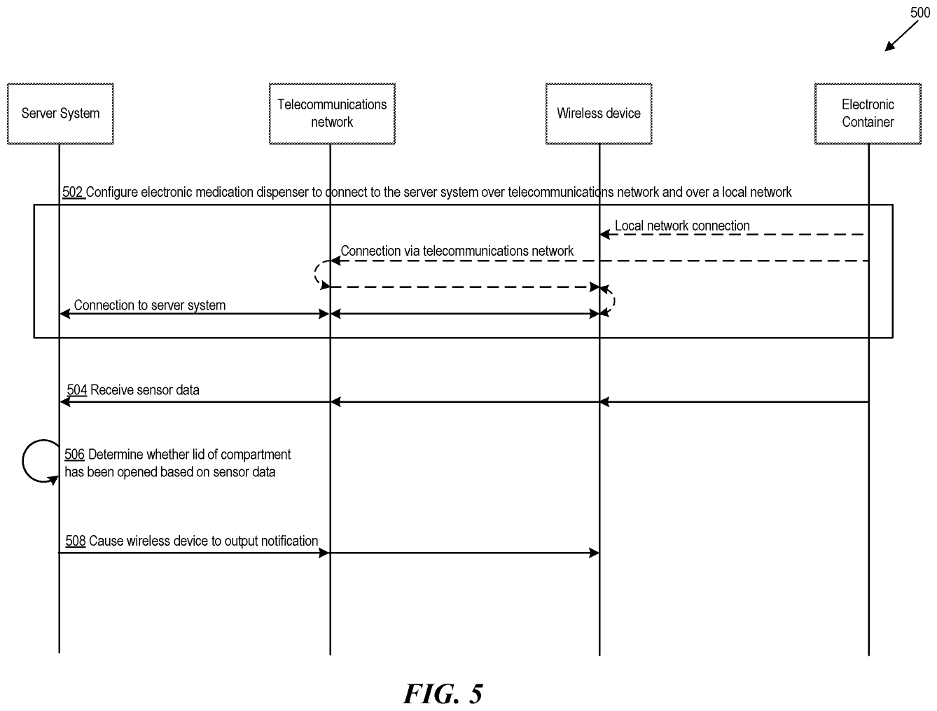

The affordance 404 can be used to track the location of the container 204 of the system 200 (e.g., as described with respect to the processes in FIG. 5). For example, the wireless device can display a GUI including the location information of the container in response to a user input on the affordance 404. In response to a user input on the affordance 406, the wireless device can display the GUI 400-2 illustrated in FIG. 4B, and in response a user input on the affordance 410, the wireless device can display the GUI 400-3 illustrated in FIG. 4C. In response to a user input on the affordance 408, the wireless device can display a GUI including notifications, and in response to a user input on the affordance 414, the wireless device can display a GUI including settings associated with the software application.

In FIG. 4B, the GUI 400-2 includes a portion 416 for generating a schedule for the consumption of a particular medicine (e.g., aspirin). The GUI 400-2 can include multiple affordances for defining actions taken by the wireless device to help a patient to follow his or her medication regime. For example, the GUI 400-2 can include an affordance for defining how many times a reminder is provided to a user, when the reminder is provided, how many dosages of the particular medication are to be taken during a day, what time the particular medication is to be taken, and duration and frequency for the scheduled alerts (e.g., including a start date and duration).

In FIG. 4C, the GUI 400-3 includes a portion for adding contact information associated with particular persons and/or parties as well as for displaying the contact information. The contact information can be used to communicate with the particular persons (e.g., caregivers) or parties (e.g., healthcare providers and/or pharmacies). The wireless device can be configured to provide messages to the particular persons and/or parties based on the stored contact information. For example, when the server system 202 in FIG. 2 determines that no medication has been taken from the container 204 at a particular time based on sensor data received from the container 204, the wireless device 206 can provide an indication to the wireless device. The wireless device 206 can display a notification on the GUI 400 (e.g., on the portion 402). Additionally, the wireless device 206 can send an indication (e.g., by a text message) to a caregiver whose contact information is stored on the wireless device. In this way, the caregiver receives a timely notification that the patient has not taken his or her medication.

FIG. 5 is a flow diagram that illustrates processes 500 for the management of medication dispensing. The processes 500 can be performed by a system including a server system (e.g., the server system 202 described with respect to FIG. 2) that is in communication with an electronic medication dispenser (e.g., the container 204 in FIG. 2) and one or more wireless devices (e.g., the wireless devices 206 and 208 in FIG. 2). The server system can include at least one hardware processor and at least one non-transitory memory storing instructions. For example, the server system includes the computing system 600 described with respect to FIG. 6. When the instructions are executed by the at least one hardware processor, the server system performs the processes 500.

At 502, the server system can configure the electronic medication dispenser to connect to the server system over the telecommunications network and to connect to the wireless device over a local network. The electronic medication dispenser and the wireless device can be associated with a common subscriber to a common carrier of the telecommunications network. The electronic medication dispenser and the wireless device can be configured to connect directly to each other over the local network and configured to connect to the server system on different channels over the telecommunications network. Communications from the electronic medication dispenser to the server system can be routed through or by the wireless device. For example, the communications from the electronic medication dispenser to the server system can be routed through the wireless device over the local network when the electronic medication dispenser and the wireless device are in a range of the local network. The communications from the electronic medication dispenser to the server system are routed via the telecommunications network when the electronic medication dispenser and the wireless device are not in the range of the local network.

At 504, the server system can receive sensor data generated by one or more sensors of the medication dispenser from the medication dispenser. For example, the server system 202 can receive sensor data from the container 204 in FIG. 2. The sensor data can be generated by one or more sensors (e.g., the sensors 302 in FIG. 3) of the container 204. In some implementations, the one or more sensors of the medication dispenser are selected from a temperature sensor, a GPS tracker, an image sensor, a weight sensor, an optical sensor, and a motion sensor.

The medication dispenser can include multiple openable compartments for holding medicines (e.g., the one or more compartments 306 in FIG. 3). A respective compartment can be configured to hold medicines to be consumed by a user of the medication dispenser during the particular time period. For example, a user of the medication dispenser can pre-fill the multiple compartments of the medication dispenser so that a respective compartment stores medications that should be consumed during a particular time period (e.g., morning, noon, afternoon, or evening) on a particular day. The pre-filling is done according to, for example, a prescription regime of a patient. For example, a first compartment includes a first set of medication that is to be taken between 8:00 AM and 9:00 AM on Monday, a second compartment includes a second set of medication that is to be taken between 8:00 PM and 10:00 PM on Monday, and a third compartment includes a third set of medication that is to be taken between 8:00 AM and 9:00 AM on Tuesday by the patient.

The sensor data can include first sensor data generated by a first sensor of the multiple sensors. The first sensor can detect the opening of a lid of a respective compartment of the multiple compartments of the medication dispenser. The first sensor can be an optical sensor (e.g., a UV or an IR sensor) or an image sensor (e.g., a camera). The optical sensor can be configured to detect the opening and closing of the lid of the respective compartment. The medication container can collect data with the optical sensor continuously or repetitively. The image sensor can be configured to collect image data of the respective compartment to detect whether the lid of the respective compartment is open or closed. In some implementations, the first sensor data generated by the medication dispenser is time-stamped.

At 506, the server system can determine whether the lid of the respective compartment has been opened during the particular time period based on the first sensor data. For example, the medication container forwards the first sensor data to the server system (e.g., the network interface 310 in FIG. 3). The server system can receive the first sensor data and process the data to determine whether the lid of the respective compartment has been opened during the particular time period. For example, if the respective compartment stores medications that should be consumed between 8:00 AM and 10:00 AM on a particular day, the server system can determine, based on the time-stamped first sensor data, whether the lid of the respective compartment was opened during that time period and day.

At 508, in response to a determination that the lid of the respective compartment has not been opened during the particular time period, the server system can cause the wireless device to output a notification (e.g., a message) indicating that the lid of the respective compartment has not been opened during the particular time period. For example, the server system sends instructions or an indication to the wireless device that allows a software application of the wireless device to output a notification. The notification could be an audio or a visual message (e.g., displayed on the GUI 400 described with respect to FIGS. 4A-4C). The notification can include a reminder or an alert. For example, the wireless device can be associated with the patient and the patient has forgotten to take his or her medication, based on the first sensor data. The notification can remind the patient to take the medication. Alternatively, the wireless device can be associated with a caregiver (e.g., a healthcare professional, a family member, or a friend) of the patient. The notification can operate as an alert to notify the caregiver that the patient has not taken his or her medication.

The medication dispenser can also be configured to provide a visual and/or audio notifications in response to the determination that the lid of the respective compartment has not been opened during the particular time period. For example, the server system provides instructions to the medication dispenser that cause the medication dispenser to provide the notification. The visual and/or audio notifications can be provided by output devices of the medication container (e.g., the input/output devices 304 in FIG. 3).

In some implementations, the medication dispenser is not in direct communication with the wireless device. Instead, the medication dispenser and the wireless device are in indirect communication with each other via the server system. For example, the medication dispenser and the wireless device are located at different locations so that the medication dispenser and the wireless device cannot be connected via a short-range communication protocol (e.g., Bluetooth or Wi-Fi). In some implementations, the medication dispenser and the wireless device are in direct wireless communication with each other.

In some implementations, in response to a determination that the lid of the respective compartment has been opened during the particular time period, the server system can forgo causing the wireless device to output the notification. The determination that the lid of the respective compartment has been opened during the particular time period can be considered an indication that the patient has accessed the medications and therefore there is no need to output the notification.

In some implementations, in response to a determination that the lid of the respective compartment has been opened, the server system can cause the wireless device to store a record indicating that the respective compartment has been opened during the particular time period. The record can be reviewed by the user of the wireless device. The record can allow the user to, for example, evaluate how consistently the patient has been consuming his or her medication.

In some implementations, the sensor data includes temperature sensor data generated by a temperature sensor of the medication dispenser. The system can further determine whether the medication dispenser is within a particular temperature range based on the temperature sensor data. In accordance with a determination that the medication dispenser is not at the particular temperature range, the server system can cause the wireless device to provide a notification indicating that the medication dispenser is not at the particular temperature range. For example, some medications are sensitive to a temperature and can be degraded at temperatures that are too high or too low. In such instances, the temperature sensor of the medication dispenser can allow monitoring of the temperature condition that the medication dispenser is exposed to. When the server system determines that the temperature of the medication dispenser is outside the particular temperature range, the server system can cause the wireless device to provide an alert to the user of the wireless device so that the user can take an action (e.g., to change the location of the medication dispenser to an appropriate ambient temperature). The system of the present disclosure can therefore enable maintaining the safety and quality of the medications stored in the medication dispenser.

In some implementations, the sensor data includes location data generated by a location tracker of the medication dispenser. The server system can determine a location of the medication dispenser based on the location data. The location data can include GPS tracking data generated by a GPS tracker of the medication dispenser. The location data can also include location data generated based on base stations (e.g., the base stations 102-1 through 102-4 in FIG. 1). For example, the medication dispenser is in communication with one or more base stations. The location data can include distance information from one or more of the base stations that the medication dispenser is communicating with. For example, the tracking can be based on multilateration (e.g., triangulation) of radio signals between multiple signal sources (e.g., the base stations or Wi-Fi access points) and the medication dispenser.

In some implementations, the sensor data further include weight or image data associated with the respective compartment. The server system can determine whether a user of the medication dispenser has removed the medicines from the respective compartment based on the weight or image data. For example, the medication dispenser can include a weight sensor configured to measure the weight of the medications stored inside a compartment. The weight sensor can detect the removal of the medications based on a difference between the weight measured at different time points. As another example, the medication dispenser can include an image sensor (e.g., a camera or an image array) configured to provide image data that depicts the interior space of a compartment. The image sensor can depict the removal of the medications from the compartment (e.g., a difference between an image including medications inside the compartment and an image of an empty compartment). The weight or image data can be time-stamped. The server system can therefore receive the weight data or the image data and determine whether the medications inside a compartment have been removed during a particular time period. In accordance with a determination that the user of the medication dispenser has not removed the medicines from the respective compartment, the server system can cause the wireless device to provide a notification indicating that the medicines are not removed to a user of the wireless device.

In some implementations, the sensor data can include medication supply tracking data generated by a sensor of the medication dispenser configured to track the amount of a particular type of medication stored within the medication dispenser. For example, the medication dispenser can be configured to store medications in bulk rather than in sub-daily dosages. The sensor could be, for example, a weight or image sensor. For example, based on image data generated by the image sensor, the server system can determine the type of medication and amount of the medication stored in the medication dispenser. The server system can be configured to be in communication with a wireless device associated with a medication supplier. For example, the wireless device 208 in FIG. 2 is associated with a medication supplier such as a pharmacy. The server system is configured to determine whether the amount of the particular type of medication is below a predefined threshold amount based on the medication supply tracking data. The server system can send a notification to the medication supplier to indicate whether the amount of the particular type of medication stored in the medication dispenser is below the predefined threshold amount. In some implementations, the medication supplier can automatically deliver the particular type of medication to the patient.

In some implementations, the server system is configured to be in communication with an additional wireless device. The server system can send instructions to the additional wireless device based on the received sensor data. The server system can cause the additional wireless device to perform an additional action. For example, the server system 202 is configured to be in communication with the wireless devices 206 and 208 in FIG. 2. The server system 202 can send instructions to both or either of the wireless devices 206 and 208. Accordingly, the wireless devices 206 and 208 can perform actions, such as display notifications.

In some implementations, the server system can receive an indication to cause the medication dispenser to perform a particular action from the wireless device. In response to receiving the indication, the server system can cause the medication dispenser to perform the particular action. For example, a user of the wireless device can provide a user input on a GUI (e.g., the GUI 400 described with respect to FIGS. 4A-4C). The user input includes setting up a reminder. The server system can receive an indication from the wireless device and forward the indication to the medication dispenser that causes the medication dispenser to output the indication (e.g., by the output devices 304 in FIG. 3). The reminder can be a visual indication (e.g., a light turning on) and/or an audio indication (e.g., an alert sound). The user can schedule repetitive indications to be output by the medication dispenser. For example, an indication will be output by the medication dispenser at the times that the patient is scheduled to take his or her medications.

In some implementations, the server system can determine that the lid of the respective compartment has been opened at a time that is different from the particular time period based on the first sensor data. For example, the lid can be opened by mistake or someone has intentionally opened the lid at a time that is different from the particular time. In response to the determination that the lid of the respective compartment has been opened at a time that is different from the particular time period, the wireless device can cause the wireless device to provide an alert to a user of the wireless device. Such an alert can be important, especially when it is important that the medication is taken at the particular time (e.g., to avoid overdosing).

In some implementations, the server system can be configured to be in communication with the wireless device and a smart container (e.g., the container 300 in FIG. 3). The smart container can be configured to store sensitive or valuable items, such as money, jewelry, documents, personal artifacts, artwork, or electronics.

The server system can receive sensor data generated by one or more sensors (e.g., the sensors 302 in FIG. 3) of the smart container from the smart container. The smart container can include an openable lid. The sensor data can include first sensor data generated by a first sensor of the one or more sensors. The first sensor detects opening of the lid of the smart container. The server system can determine whether the lid of the smart container has been opened based on the first sensor data. In accordance with a determination that the lid of the smart container has been opened, the server system can cause the wireless device to perform an action.

In some implementations, in response to a determination that the lid of the smart container has been opened, the server system can cause the wireless device to output a notification indicating that the lid has been opened.

In some implementations, the sensor data further include weight or image data associated with the smart container. The server system can determine whether an item (e.g., an item selected from money, jewelry, documents, personal artifacts, artwork, or electronics) has been removed from inside the smart container based on the weight or image data. In accordance with a determination that the item has been removed from the smart container, the server system can cause the wireless device to provide a user of the wireless device a notification indicating that the item has been removed.

### Computer System

FIG. 6 is a block diagram that illustrates an example of the computing system 600 in which at least some operations described herein can be implemented. As shown, the computing system 600 can include: one or more processors 602, main memory 606, non-volatile memory 610, a network interface device 612, video display device 618, an input/output device 620, a control device 622 (e.g., keyboard and pointing device), a drive unit 624 that includes a storage medium 626, and a signal generation device 630 that are communicatively connected to a bus 616. The bus 616 represents one or more physical buses and/or point-to-point connections that are connected by appropriate bridges, adapters, or controllers. Various common components (e.g., cache memory) are omitted from FIG. 6 for brevity. Instead, the computing system 600 is intended to illustrate a hardware device on which components illustrated or described relative to the examples of the figures and any other components described in this specification can be implemented.

The computing system 600 can take any suitable physical form. For example, the computing system 600 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 600. In some implementation, the computing system 600 can be an embedded computer system, a system-on-chip (SOC), a single-board computer system (SBC) or a distributed system such as a mesh of computer systems or include one or more cloud components in one or more networks. Where appropriate, one or more computer systems 600 can perform operations in real-time, near real-time, or in batch mode.

The network interface device 612 enables the computing system 600 to mediate data in a network 614 with an entity that is external to the computing system 600 through any communication protocol supported by the computing system 600 and the external entity. Examples of the network interface device 612 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 606, non-volatile memory 610, machine-readable medium 626) can be local, remote, or distributed. Although shown as a single medium, the machine-readable medium 626 can include multiple media (e.g., a centralized/distributed database and/or associated caches and servers) that store one or more sets of instructions 628. The machine-readable (storage) medium 626 can include any medium that is capable of storing, encoding, or carrying a set of instructions for execution by the computing system 600. The machine-readable medium 626 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 610, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 604, 608, 628) set at various times in various memory and storage devices in computing device(s). When read and executed by the processor 602, the instruction(s) cause the computing system 600 to perform operations to execute elements involving the various aspects of the disclosure.

### Remarks

The terms “example,” “embodiment,” and “implementation” are used interchangeably. For example, references to “one example” or “an example” in the disclosure can be, but not necessarily are, references to the same implementation; and, such references mean at least one of the implementations. The appearances of the phrase “in one example” are not necessarily all referring to the same example, nor are separate or alternative examples mutually exclusive of other examples. A feature, structure, or characteristic described in connection with an example can be included in another example of the disclosure. Moreover, various features are described which can be exhibited by some examples and not by others. Similarly, various requirements are described which can be requirements for some examples but no other examples.

The terminology used herein should be interpreted in its broadest reasonable manner, even though it is being used in conjunction with certain specific examples of the invention. The terms used in the disclosure generally have their ordinary meanings in the relevant technical art, within the context of the disclosure, and in the specific context where each term is used. A recital of alternative language or synonyms does not exclude the use of other synonyms. Special significance should not be placed upon whether or not a term is elaborated or discussed herein. The use of highlighting has no influence on the scope and meaning of a term. Further, it will be appreciated that the same thing can be said in more than one way.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import can refer to this application as a whole and not to any particular portions of this application. Where context permits, words in the above Detailed Description using the singular or plural number may also include the plural or singular number respectively. The word “or” in reference to a list of two or more items covers all of the following interpretations of the word: any of the items in the list, all of the items in the list, and any combination of the items in the list. The term “module” refers broadly to software components, firmware components, and/or hardware components.

While specific examples of technology are described above for illustrative purposes, various equivalent modifications are possible within the scope of the invention, as those skilled in the relevant art will recognize. For example, while processes or blocks are presented in a given order, alternative implementations can perform routines having steps, or employ systems having blocks, in a different order, and some processes or blocks may be deleted, moved, added, subdivided, combined, and/or modified to provide alternative or sub-combinations. Each of these processes or blocks can be implemented in a variety of different ways. Also, while processes or blocks are at times shown as being performed in series, these processes or blocks can instead be performed or implemented in parallel or can be performed at different times. Further, any specific numbers noted herein are only examples such that alternative implementations can employ differing values or ranges.

Details of the disclosed implementations can vary considerably in specific implementations while still being encompassed by the disclosed teachings. As noted above, particular terminology used when describing features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed herein, unless the above Detailed Description explicitly defines such terms. Accordingly, the actual scope of the invention encompasses not only the disclosed examples, but also all equivalent ways of practicing or implementing the invention under the claims. Some alternative implementations can include additional elements to those implementations described above or include fewer elements.

Any patents and applications and other references noted above, and any that may be listed in accompanying filing papers, are incorporated herein by reference in their entireties, except for any subject matter disclaimers or disavowals, and except to the extent that the incorporated material is inconsistent with the express disclosure herein, in which case the language in this disclosure controls. Aspects of the invention can be modified to employ the systems, functions, and concepts of the various references described above to provide yet further implementations of the invention.

To reduce the number of claims, certain implementations are presented below in certain claim forms, but the applicant contemplates various aspects of an invention in other forms. For example, aspects of a claim can be recited in a means-plus-function form or in other forms, such as being embodied in a computer-readable medium. A claim intended to be interpreted as a mean-plus-function claim will use the words “means for.” However, the use of the term “for” in any other context is not intended to invoke a similar interpretation. The applicant reserves the right to pursue such additional claim forms in either this application or in a continuing application.

I/We claim:

1. A server system associated with a telecommunications network, the server system being configured to communicate with a wireless device and an electronic medication dispenser, the server system comprising:
at least one hardware processor; and
at least one non-transitory memory storing instructions, which, when executed by the at least one hardware processor, cause the server system to:
configure the electronic medication dispenser to connect to the server system over the telecommunications network and to connect to the wireless device over a local network,
wherein the electronic medication dispenser and the wireless device are associated with a common subscriber to a common carrier of the telecommunications network,
wherein the electronic medication dispenser and the wireless device are configured to connect directly to each other over the local network and configured to connect to the server system on different channels over the telecommunications network, and
wherein communications from the electronic medication dispenser to the server system are routed through or by the wireless device;
receive, from the electronic medication dispenser, sensor data generated by one or more sensors of the electronic medication dispenser,
wherein the electronic medication dispenser includes multiple openable compartments configured for holding medicines,
wherein the sensor data includes first sensor data generated by a first sensor of the multiple sensors, and
wherein the first sensor detects a position of a lid of a respective compartment of the multiple compartments of the electronic medication dispenser;
determine, based on the first sensor data, whether the lid of the respective compartment has been opened during a particular time period,
wherein the respective compartment is configured to hold medicines to be consumed by a user of the electronic medication dispenser during the particular time period;
in response to a determination that the lid of the respective compartment has not been opened during the particular time period,
cause the wireless device to output a notification indicating that the lid of the respective compartment has not been opened during the particular time period.

2. The server system of claim 1, further caused to:
in response to a determination that the lid of the respective compartment has been opened during the particular time period,
forgo causing the wireless device to output the notification.

3. The server system of claim 1, further caused to:
in response to a determination that the lid of the respective compartment has been opened,
cause the wireless device to store a record indicating that the respective compartment has been opened during the particular time period.

4. The server system of claim 1, wherein:
the sensor data comprises temperature sensor data generated by a temperature sensor of the electronic medication dispenser, and
the system is further caused to:
determine, based on the temperature sensor data, whether the electronic medication dispenser is within a particular temperature range, and
in accordance with a determination that the electronic medication dispenser is not at the particular temperature range,
cause the wireless device to provide a notification indicating that the electronic medication dispenser is not at the particular temperature range.

5. The server system of claim 1, wherein:
the sensor data comprises location data generated by a location tracker of the electronic medication dispenser, and
the system is further caused to:
determine, based on the location data, location of the electronic medication dispenser.

6. The server system of claim 1, wherein:
the server system is configured to be in communication with an additional wireless device,
the server system is caused to send, based on the received sensor data, instructions to the additional wireless device that cause the additional wireless device to perform an additional action.

7. The server system of claim 1, wherein:
the sensor data further comprises weight or image data associated with the respective compartment, and
the server system is further caused to:
determine, based on the weight or image data, whether a user of the electronic medication dispenser has removed the medicines from the respective compartment, and
in accordance with a determination that the user of the electronic medication dispenser has not removed the medicines from the respective compartment,
cause the wireless device to provide, to a user of the wireless device, a notification indicating that the medicines are not removed.

8. The server system of claim 1, wherein:
the one or more sensors of the electronic medication dispenser are selected from a temperature sensor, a global positioning system (GPS) tracker, an image sensor, a weight sensor, an optical sensor, and a motion sensor.

9. The server system of claim 1, further caused to:
receive, from the wireless device, an indication to cause the electronic medication dispenser to perform a particular action,
in response to receiving the indication,
cause the electronic medication dispenser to perform the particular action.

10. The server system of claim 1, further caused to:
determine, based on the first sensor data, that the lid of the respective compartment has been opened at a time that is different from the particular time period, and
in response to a determination that the lid of the respective compartment has been opened at a time that is different from the particular time period,
cause the wireless device to provide, to a user of the wireless device, an alert.

11. The server system of claim 1, wherein:
the electronic medication dispenser is configured to provide a visual or audio notification in response to the determination that the lid of the respective compartment has not been opened during the particular time period.

12. The server system of claim 1, wherein:
the sensor data comprises medication supply tracking data generated by a sensor of the electronic medication dispenser configured to track an amount of a particular type of medication stored within the electronic medication dispenser,
the server system is further configured to be in communication with a wireless device associated with a medication supplier, and
the system is further caused to:
determine, based on the medication supply tracking data, whether the amount of the particular type of medication is below a predefined threshold amount, and
send a notification to the medication supplier to indicate whether the amount of the particular type of medication stored in the electronic medication dispenser is below the predefined threshold amount.

13. The system of claim 1, wherein:
the electronic medication dispenser is not in direct communication with the electronic device.

14. The system of claim 1, wherein communications from the electronic medication dispenser to the server system are:
routed through the wireless device over the local network when the electronic medication dispenser and the wireless device are in a range of the local network, and
routed via the telecommunications network when the electronic medication dispenser and the wireless device are not in the range of the local network.

15. A server system configured to be in communication with a wireless device and a smart container, the server system comprising:
at least one hardware processor; and
at least one non-transitory memory storing instructions which, when executed by the at least one hardware processor, cause the server system to:
receive, from the smart container, sensor data generated by one or more sensors of the smart container,
wherein the smart container includes an openable lid,
wherein the sensor data includes first sensor data generated by a first sensor of the one or more sensors, and
wherein the first sensor detects opening of the lid of the smart container;
determine, based on the first sensor data, whether the lid of the smart container has been opened,
in accordance with a determination that the lid of the smart container has been opened,
cause the wireless device to perform an action.

16. The server system of claim 15, wherein:
in response to a determination that the lid of the smart container has been opened,
cause the wireless device to output a notification indicating that the lid has been opened.

17. The server system of claim 15, wherein:
the sensor data comprises location data generated by a location tracker of the smart container, and
the system is further caused to:
determine, based on the location data, location of the smart container.

18. A method performed by a server system for managing medication dispensing, the method comprising:
receiving, by the server system from an electronic medication dispenser, sensor data generated by one or more sensors of the electronic medication dispenser,
wherein the electronic medication dispenser includes multiple openable compartments for holding medicines,
wherein the sensor data includes first sensor data generated by a first sensor of the multiple sensors, and
wherein the first sensor detects the opening of a lid of a respective compartment of the multiple compartments of the electronic medication dispenser;
determining, by the server system based on the first sensor data, whether the lid of the respective compartment has been opened during a particular time period,
wherein the respective compartment is configured to hold medicines to be consumed by a user of the electronic medication dispenser during the particular time period;
in response to a determination that the lid of the respective compartment has not been opened during the particular time period,
causing, by the server system, a wireless device to output a notification indicating that the lid of the respective compartment has not been opened during the particular time period.

19. The method of claim 18, further comprising:
in response to a determination that the lid of the respective compartment has been opened during the particular time period,
forgoing, by the server system, causing the wireless device to output the notification.

20. The method of claim 18, further comprising:
in response to a determination that the lid of the respective compartment has been opened,
causing, by the server system, the wireless device to store a record indicating that the respective compartment has been opened during the particular time period.
